(19) United States
(12) Patent Application Publication
(10) Pub. No.: US 2009/0234201 A1
(43) Pub. Date: Sep. 17, 2009

(54) Belt integrated with stress sensing and output reaction

(76) Inventors: Jung-Tang Huang, Taipei (TW); Chun-I Sun, Taipei (TW)

Correspondence Address: Jung-Tang Huang, 5F., No.7, Lane 10, Sec, 2, Bade Rd.,, Da-an District, Taipei City, 106, TW

(21) Appl. No.: 12/381,456

(22) Filed: Mar. 13, 2009

(30) Foreign Application Priority Data
Mar. 12, 2008 (TW) TW097108612

(51) Int. Cl. A61B 5/00

(52) U.S. Cl. 600/301

(57) ABSTRACT

This invention disclosed a method for monitoring the status of health by way of a belt which is integrated with stress sensing sensor and reaction output function. The user could follow its assistant to do breathing exercises, like belly breathing.

### BACKGROUND

1. Field of Invention

This invention relates to wearing type sensing and interactive device and a method for health monitoring and exercise assistance. It specifically relates to a binding belt associated with pressure or other sensing device, which includes user interface to output the response signal, to perform health monitoring and exercise assistance.

2. Description of Related Arts

Electronic game is a commonly interactive device, and interaction and control can be performed in association with force feedback; however, it does not have training mechanism of physiological function accompanied with breathing, and it is only pure reflective type interaction; moreover, it can not perform activity or health consultation through monitoring way. Pathological detection device such as blood pressure detector can detect physiological status, but it can not be carried at any time with ease so as to perform continuous monitoring in long term; moreover, the user can not, based on the response signal of the blood pressure meter, perform will interaction so as to achieve the purpose of blood pressure regulation. Exercise type heart beat tracer can be worn for a long term to trace the heart beat, but the user has to monitor by himself/herself and no relative activity suggestion will be sent out, instead, the user has to do independent judgment and take action.

Take breathing as an example, we know that breathing is the most basic way our body interacts with the world. Smooth breathing will supply sufficient oxygen to the blood to support body's use. Breathing can be divided into chest breathing and belly breathing. During chest breathing, chest will wave up and down and most of the air enters the upper half of lung; chest breathing is good after exercise because at that time, body will need oxygen urgently. During belly breathing, when you inhale, the belly will get raised, on the contrary, when you exhale, the belly will get dented naturally; such kind of breathing only consume very small amount of energy and can reinforce the air exchange at the lower half of lung. When the body is not at severe exercise, belly breathing is more suitable. However, general people usually do not monitor their own breathing way and normal breathing method; therefore, it is necessary to provide an innovative tool to guide the user to have correct breathing habit.

In general, the belly breathing has many advantages, for example, it will increase the pressure of the belly, massage the organs, and it will distribute the blood of the belly very well; meanwhile, it will oppress gradually the activity of sympathetic nerve (in charge of exciting function) of the autonomic nerve and in the mean time, the parasympathetic nerve function (relaxing function) will be gradually reinforced; after continuous practice for a period of time, it can cure fatigue and waist and back ache, etc. However, to get used to belly breathing, it takes correct method: You can have your body lie flat or sit or stand, put both of your hands on the upper side of your belly so as to feel the raised or dented belly; try to keep yourself relaxed, and the major goal is to relax both your body and mind. When you inhale, your belly will get raised (Put a little force to the waist during the inhalation), and when you exhale, the belly will get dented naturally. During the breathing, relax the breast muscle, do not swing your shoulder upwards and downwards and pay your attention to feel your own breathing. Appropriate breathing frequency is 4-6 times each minute (that is, inhalation four times and exhalation four times); one breathing will take 10 to 15 seconds; it is suggested to make smooth inhalation and exhalation and do not use too much force, that is, the better situation is, the exhalation takes about two times that of inhalation (that is, exhalation is slower than inhalation). During the breathing, it is good to make large and deep breath, and it is OK to use either nose or mouth for the breathing. It is best to do the practice 10 minutes after getting up in the morning and 10 minutes before going to bed; you can slowly extend the time, but it is better not to exceed one hour.

Since belly breathing needs more practice, currently, there is no tool that that can guide people to learn belly breathing easily; moreover, there is no wearable tool to help people to monitor in time and in the long term and to provide interactive suggestion to the user for the health status and exercise status.

### SUMMARY

The main objective of this invention is to provide in time and long term monitoring and to provide interactive suggestion to the user for health status and exercise status so that the user can control the action through the will and improve the health status.

During regular dining process, over-diet is usually not noticed; therefore, how to perform long term monitoring and how to provide reminding signal in time to stop the over-diet from the user is the second objective of this invention.

During the exercise, can the breathing be done in accordance with the rhythm of the exercise, or during the belly breathing, can the period be controlled appropriately, or is the depth of the breathing is enough, etc., that is, the exercise assistance function is also one of the objectives of this invention. The exercise assistance ways are done as in the followings: First, the periodic exercise command is sent out every day to guide gradually the user to breathe accordingly and to perform in time activity monitoring; furthermore, based on the user's capability, appropriate and correct breathing period is guided slowly, then the practicing time is increased day by day until user has a belly breathing habit.

### BRIEF DESCRIPTION OF DRAWINGS

The detailed drawings of this invention will be fully understood from the following descriptions wherein:

FIG. 1 is an embodiment assembly drawing of belt in accordance with the present invention.

FIG. 2 is a partial structure drawing of the embodiment (not including upper cover) of belt in accordance with the present invention.

FIG. 3 is a structural cross section of the belt embodiment of the present invention.

FIG. 4 is an assembly illustration of the second embodiment of belt in accordance with the present invention.

FIG. 5 is a structural cross sectional drawing of the second embodiment of belt in accordance with the present invention.

FIG. 6 is a structural drawing of waist belt locking head (with back cover removed) in accordance with the present invention.

FIG. 7 is a cover of waist belt locking head in accordance with the present invention.

FIG. 8 is the cross section of waist belt locking head in accordance with the present invention.

FIG. 9 is a system block diagram of the present invention.

FIG. 10 is cross section of array type stress sensor in accordance with the present invention.

FIG. 11 is an illustration of the embodiment of binding belt in accordance with the present invention.

FIG. 12 is a utilization illustration of binding belt in accordance with the present invention.

### DETAILED DESCRIPTION OF THE PREFERRED EMBODIMENT

In order to simplify the description of the present invention, the waist belt embodiment will be used, in fact, the present invention can also be used in the similar structures such as: wrist protection device, knee protection device, gloves, sleeve, socks and trousers so as to monitor the muscle exercise within body of different object to be monitored and to perform interaction.

### Embodiment 1

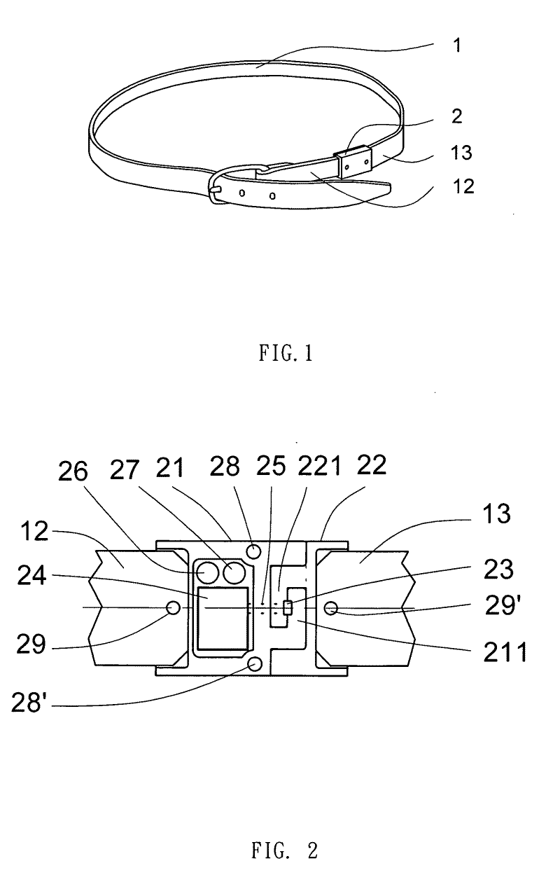

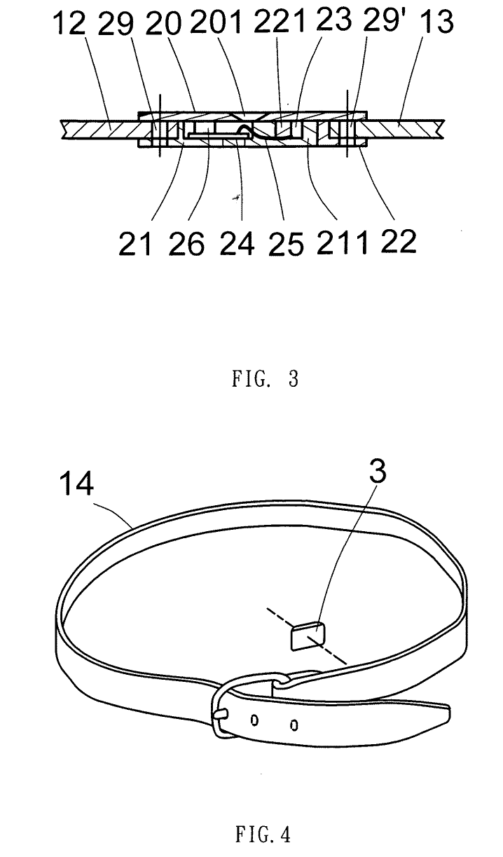

Referring to FIG. 1 to FIG. 3, this embodiment associates sensor 2 with waist belt 1. Waist belt 1 can be divided into waist belt left end 12 and waist belt right end 13, and sensor 2 is connected to between left and right ends through fixing holes 29 and 29′. Sensor 2 is formed respectively by sensor left component 21, sensor right component 22 and cover plate 20. Moreover, left and right components are hooked to each other by left component tension hook 211 and right component tension hook 221, between, a stress sensor 23 is clamped in between; the pressure sensing result is then transferred through connection wire 25 to printed circuit board component 24 within sensor left component 21, and printed circuit board component 24 includes the software and hardware system of entire circuit and the power supply of the battery. Within sensor left component 21, other sensor 26 is also installed, and, according to the need, it can be sensors for sound, temperature, ultrasonic wave, vibration, acceleration or light, etc. Human-machine user interface or the message transfer among devices is done through output and input interface 27, based on the need, different output and input devices can be selected, for example, button, vibration device, speaker and other sound device, light emitting diode (LED) or wired and wireless transfer interface, etc. Cover plate 20 is fixed, through fixing hole 201, to fixing screw hole 28, 28′. Meanwhile, right component tension hook 221 is inhibited to separate from left component tension hook 211 through the upper side of cover plate 20.

During the application, waist belt 1 is worn at the upper side of the trouser through general trouser waist belt way. During normal operation, the following steps are implemented:
Step 1: Sensor 2 will sense the tension at the waist of the user, and the pressure change generated will be responded to stress sensor 23 to generate in time and relative response signal;
Step 2: Convert the response signal from stress sensor 23 through electronic circuit, then compare it with the recorded status (for example, the exceeding of the average upper limit and the exceeding of the periodic operation are distinguished) and update the record to generate analysis status.
Step 3: The human-machine interface or signal output device will send out corresponding correction notification signal (For example, if the exceeding of the average upper limit is found, over-diet signal will be sent out).
Step 4: The user will then perform behavior improvement interaction according to the correction notification signal sent out (for example, the stop of taking food, etc.) so as to improve the waist tension of the user. And finally, the in-time control of the diet amount will be achieved.

When it is worn for a long time accompanied with the above circulated control steps, the engorgement habit of the user will be gradually improved, and the health situation of the user will be improved accordingly.

During the special function operation, for example, the belly breathing training, and under normal operation, through output and input interface 27 change system, the following steps will be implemented:
Step 1: Sensor 2 will sense the tension change magnitude and tension change period of the waist along with breath, and the generated pressure change will be responded on stress sensor 23 to generate in time and corresponding response signal;
Step 2: A circuit will be used to convert the response signal of stress sensor 23 and compare it with the recorded status (for example, the upper and lower limit trend and periodic operation, etc.) and update the record to generate analysis status.
Step 3: The signal output and input interface 27 will respond to the in time and relative response signal generated by the breath and send out corresponding guiding signal. When the period is too short, the device will assist and guide the breath slower. That is, it will respond in one hand, and lengthen the guiding signal in another hand; when the breath is too shallow, the device will assist and guide to increase the depth of the breath. That is, it will respond in one hand, and gradually increase the guiding signal in another hand.
Step 4: If the user can act in accordance with the guiding signal and sensor 2 can sense that the tension change magnitude and tension change period of the waist along with breath can meet the setting and the training time setup value is achieved as well, then a completion notification signal will be sent out to finish belly breathing training activity.

The entire step is similar to the practice in yoga, that is, the user should act in accordance with the oral command of the instructor to follow gradually. Through system operation and automatic scheduled startup, accompanied with long term belly breathing training, the health status of the user can then be improved. The way belly breathing can improve health is as that in the book of “Belly breathing, a health-building method through Qigong: The strongest disease-cure, health and long life method” (Written by Hsien-Min Chao and printed by Aniston Publishing Center), that is, it takes long term of practice. Moreover, in the present invention, through periodic reminding, monitoring and guiding of the device and system, as long as the user act in accordance with it, the expected health purpose is naturally and easily achieved.

For the application of the binding belt for other parts, for example, in the first exercise and ball striking activities, the binding belt can be installed on body part that should exert appropriate force, for example, the front arm, wrist, elbow, upper arm, shank and thigh, etc. That is, interactive learning can be performed on, for example, the force-exerting point, force-exerting depth and the force-exerting explosion period of the muscle. Accompanied with the response from different sensor 26, the exerting force can be improved in real time and in interactive way so that best effect can be generated in the exertion of action and force; furthermore, fast and correct learning effect can be generated to facilitate the correction of exercise error and to reduce exercise injury.

For the application in jogging, vibration sensor 26 can be used together to perform step counting and breathing action combination detection, interactive monitoring and consultation.

### Embodiment 2

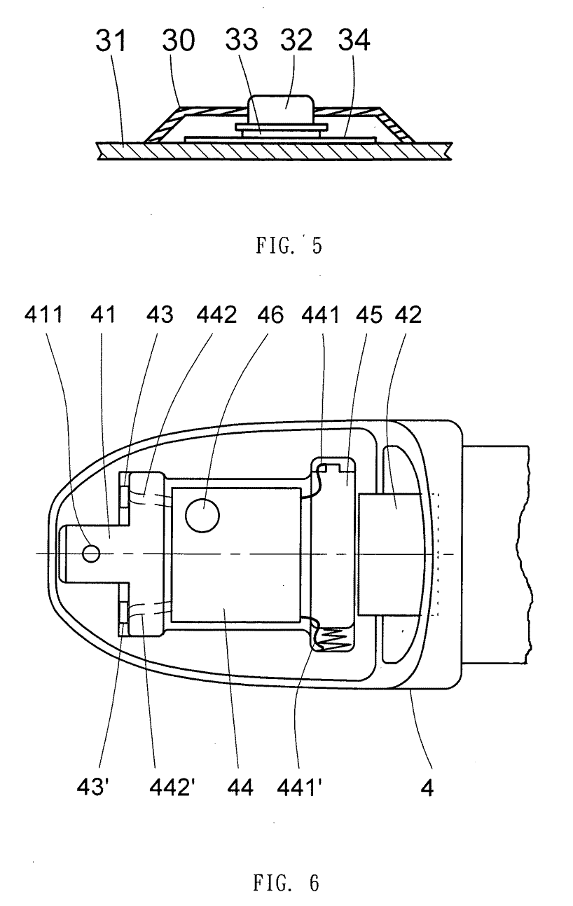

Referring to FIG. 4 and FIG. 5, they illustrate an embodiment of the present invention that combines sensor 3 and waist belt 14 in the inner side. Sensor 3 is formed by printed circuit board component 34 and stress sensor 33 enclosed by outer shell 30 and waist belt 31. Stress button 32 is in between stress sensor 33 and outer shell 30 to transfer the waist pressure directly to stress sensor 33. Printed circuit board component 34 includes the software and hardware system of entire circuit, the supply of battery power and output and input interface. Its function and operation procedure is the same as embodiment 1.

### Embodiment 3

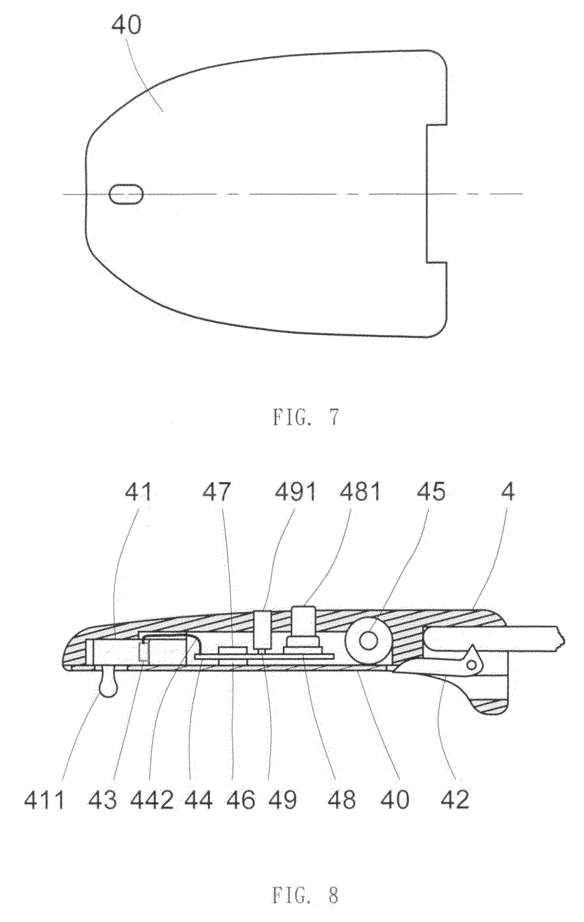

Referring to FIG. 6 to FIG. 8, in the present embodiment, sensor 4 and locking head of waist belt are combined into one piece, and the waist belt association way is the same as the locking head of general waist belt, that is, tooth press 42 will clamp waist belt. Another end of waist belt is hooked at different hole positions according to the waist size by cylindrical hook head 411. The locking head sensor 4 of waist belt includes T block 41 with stress sensor 43, 43′ clamped in between. When the tension of waist belt is transferred from cylindrical hook head 411 to the T block 41, it will create clamping force on the sensors 43, 43′ above it; tensions of different magnitudes will generate indirectly response signals of different sizes, and the signals will then be transferred to printed circuit board component 44 through connection wires 442, 442′. Printed circuit board component 44 has power supplied by battery 45 through power wires 441, 441′. Above it includes sensor 46, which can be formed by sensors such as sound, temperature, ultrasonic wave, vibration, etc. so as to satisfy different operation function needs. Message transfer between human and machine or among devices can be done through output and input interface 47; meanwhile, based on the needs, output and input devices such as vibration device, speaker and sound device, or wired and wireless transfer interface, etc. can be selected. Above circuit switch 48 is connected with button 481 to transfer the user operation message to printed circuit board component 44. The response signal can be transferred through light emitting diode 49 and optical guiding column 491 to sensor 4 located at the exterior side of locking head type outer shell of the waist belt. After the assembly, the entire structure will be covered by cover plate 40 from the inner side.

Although the structure of embodiment 3 is different than that of embodiments 1 and 2, yet its operational function, step and result is still the same as embodiment 1.

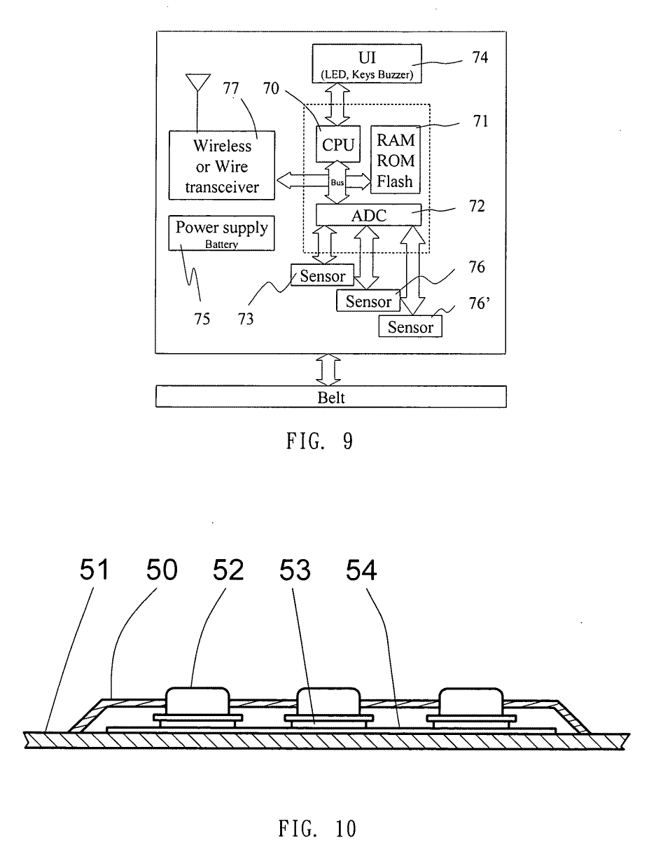

Meanwhile, the comparison of the entire system structure shows the same. Referring now to system block diagram in FIG. 9, binding belt such as waist belt is the source of the pressure or tension of stress sensor 73. When the object to be tested is limited to binding belt region of certain cross sectional area, as long as the muscle of the body of the person to be tested exerts force, an outward-going stress will then be sensed at sensor 73. This response signal will be converted by analog digital conversion interface 72 into digital signal to be transferred to microprocessor 70 for analysis; the analysis program will be installed in memory 71 for execution, and the analysis result will be recorded in memory 71 too. Meanwhile, user interface 74 will perform system operation control; when there are other needs, for example, the performance of breathing monitoring in accordance with walking, other sensors 76, 76′ can be accompanied, for example, sensors such as sound, temperature, ultrasonic and vibration, etc. When there is remote control need, it can be accompanied with wired or wireless output and input interface 77 to perform signal transfer or remote monitoring. And the entire power consumption is supplied by battery 75.

### Embodiment 4

Referring to FIG. 10, when there is muscle stress of large area that needs to be performed with in time and interactive monitoring, for example, the binding belt applied on the thigh, or when force-exerting situations of several muscles are to be monitored respectively, for example, the wrist and finger control as controlled by several muscles on the front arm, array arrangement of stress sensor 53 on printed circuit board component 54 can be adopted. Stress button 52 is arranged on each sensor 53 in one to one basis, with outer side covered by outer shell 50. And it combines with binding belt 51 to form one body.

### Embodiment 5

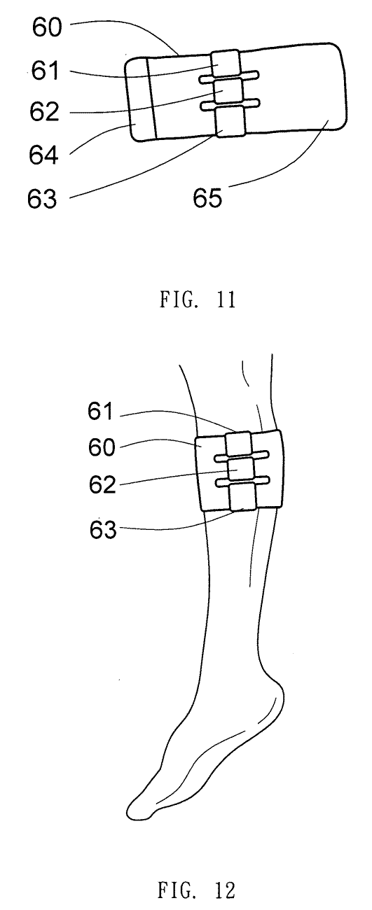

Please also refer to FIG. 11 and FIG. 12, when it is to cope with non-flat muscle surface curvature of large area, binding belt 60 with separately arranged stress sensor 61, 62, 63 will be adopted. The way it is tied is like the knee protecting device that is enclosed and tied by Nylon Fastener 64, 65. By doing so, real time muscle interaction and monitoring on multiple curved surfaces can then be achieved.

Although the invention has been described with reference to particular embodiments, the description is only an example of the invention's application and should not be taken as a limitation. Various adaptations and combinations of features of the embodiments disclosed are within the scope of the invention as defined by the following claims.

What is claimed is:

1. A binding belt containing sensor comprising mainly of waist belt, wrist protector, knee protector, gloves, sleeve, socks, trouser or similar structure; it is then accompanied with electronic circuit, power and at least one type of stress sensor; furthermore, it is associated with human-machine interface or signal output device to be used in continuous and long time tracking of the stress response signal within the body of the object to be tested that is tied tightly by the binding belt; response signal then is converted by circuit and software, and corresponding change notification signal is sent out by human-machine interface or output device; therefore, the person under test can, based on the output change notification signal, perform in time interaction through will, change the body operation status.

2. The binding belt of claim 1 wherein the stress sensor is pressure or tension sensor; in addition, step counter, temperature sensor, sound sensor, current sensor, vibration sensor or image sensor are used together.

3. The binding belt of claim 1 wherein the human-machine interface is switch, button, LED, LCD, buzzer, speaker or a combination of several above items.

4. The binding belt of claim 1 wherein signal output device is wireless transmission device, wired transmission device or a combination of both; the signal receiving device receives response signal or provide control signal.

5. The binding belt of claim 1 wherein stress sensor is a single sensor or multiple sensors arranged in array way.

6. A method for exercise status tracking, health status tracking or assistance to health, and the method uses at least one stress sensor binding belt to implement the following steps:
(a) The relative position of binding belt fixed sensor and the object to be tested;
(b) Under normal operation condition, the sensor senses the stress operation within the object to be tested, and the measured pressure or tension change is used to generate corresponding response signal;
(c) An electronic circuit converts the response signal of the sensor, then through human-machine interface or signal output device, corresponding change notification signal is sent out;
(d) The person under test, based on the change notification signal sent out, performs in time interaction through will so as to improve the operation status within the body.

7. The method of claim 6 wherein, based on the response signal transferred, the motion of the object to be tested, whether the force exertion at relative time for the object to be tested is appropriate or whether the magnitude of the force exerted has reached the setup standard for the object to be tested, is traced, and corresponding change notification signal is sent out to be used as indicator in the learning program.

8. The method of claim 6 wherein, based on the response signal transferred, whether a negative growth can be seen on the object to be tested or a critical value has been reached, is traced, and corresponding change notification signal is sent out so as to be used as indicator for health status.

9. The method of claim 6 wherein when the binding belt is tied to the belly, based on the response signal transferred, it detects whether the belly breathing method has been reached effectively, and corresponding change notification signal is sent out to assist the reach of the training purpose of performing belly breathing method.

10. The method of claim 9 wherein to act in accordance with the signal from the step counter, corresponding change notification signal is sent out so as to reach the purpose of coordinating belly breathing and the step rhythm.

11. The method of claim 6 wherein to act in accordance with the signal from the pulse sensor, corresponding change notification signal is sent out so as to reach the purpose of assisting the control of the exercise rhythm.

12. The method of claim 6 wherein to act in accordance with the signal from the sound sensor, corresponding change notification signal is sent out so as to reach the purpose of detecting the infant's heart beat within the belly of the pregnant person and the rhythm of the person under test.
